(19) United States
(12) Patent Application Publication
(10) Pub. No.: US 2011/0202014 A1
(43) Pub. Date: Aug. 18, 2011

(54) Adapter device for application of small amounts of fat graft material by use of syringes

(76) Inventor: Till Sebastian Mutzbauer, Zuerich (CH)

(21) Appl. No.: 12/705,594

(22) Filed: Feb. 13, 2010

(51) Int. Cl. A61M 5/31

(52) U.S. Cl. 604/241

(57) ABSTRACT

The present invention provides a device and the method of use for the delivery of fat graft material to treat delicate anatomical areas. By use of the device and method very low amounts of fat can be administered and easily be controlled. This can be achieved by connecting the fat graft syringe assembly to a conventional delivery tubing system normally used for e.g. the controlled, low-volume application of local anesthetics. The driving force to move the fat particles through the syringe and the attached cannula is generated by a flow of normal saline solution which is triggered by the surgeon himself by use of a foot switch activating the electronic delivery device.

### BACKGROUND OF THE INVENTION

The invention relates generally to a fat graft syringe assembly, and method for the delivery of fat graft material to treat a surgical site.

In the surgical area of the face, it is a common esthetic surgical procedure to administer fat graft material to anatomical structures below the facial skin.

Unfortunately, the currently available devices designed for delivering fat to delicate areas of the facial skin suffer from the inability to administer very small fat particles under the skin. A very low size of fat particles can be achieved by a special harvesting process of fat grafts from donor sites of the human body. To readminister the fat, low volume size syringes are available. Once the cannula is placed underneath the patient's skin, movement of the syringe with the attached cannula is necessary during the application of the fat, as the amount of fat administered to a specific area has to be kept as small as possible.

The surgeon, however, has to simultaneously inject the fat by applying pressure on the syringe piston while he is directing the syringe with the attached cannula through the tissue. Thereby, a controlled delivery of the fat—especially of small amounts—is difficult.

Accordingly, the invention provides embodiments of devices and methods of use for the delivery of fat graft material to treat even delicate anatomical sites, that overcome the disadvantages of known devices and methods while offering features not present in known art.

The disadvantages of conventionally available systems are not necessarily recognized or known as such.

Although certain deficiencies in the related art are described in this background discussion and elsewhere, it will be understood that these deficiencies were not necessarily heretofore recognized or known as deficiencies. Furthermore, it will be understood that, to the extent that one or more of the deficiencies described herein may be found in an embodiment of the claimed invention, the presence of such deficiencies does not detract from the novelty or non-obviousness of the invention or remove the embodiment from the scope of the claimed invention.

### SUMMARY OF THE INVENTION

1. The invention, according to one embodiment, is a fat graft syringe assembly for delivering fat graft material. The fat graft syringe assembly comprises a syringe subassembly including a syringe adapter coupled to a syringe. The adapter consists of a proximal portion and a distal portion; the distal portion can be coupled to a 1 ml syringe by a conical fitting, the proximal portion of the adapter has an external luer-lock thread that can be affixed to an internal luer-lock thread of a conventional infusion or local anesthetic delivery tubing system
2. Another embodiment is a fat graft syringe assembly for delivering fat graft material, wherein the proximal portion of the adapter has an internal luer-lock thread that can be affixed to an external luer-lock thread of a conventional infusion delivery tubing system
3. Yet another embodiment is a graft syringe, wherein the syringe adapter is included in the 1 ml syringe itself serving as its proximal connecting portion which has a size equal to an external luer-lock thread. Thereby, a conventional delivery tubing system with its distal internal luer-lock thread can be connected directly to the proximal portion of the syringe.
4. The infusion or anesthetic delivery tubing system attached to the syringe assembly is filled with normal saline. Preferably, the flow of normal saline is achieved by a trigger mechanism activated by use of a foot switch.

### BRIEF DESCRIPTION OF THE DRAWINGS

The present invention can be better understood by reading the following descriptions of the presently preferred embodiments together with the accompanying drawings, in which

FIG. 1 is a side sectional view of the fat graft syringe assembly in accordance with embodiment 1 of the invention

FIG. 2 is a side sectional view of the the fat graft syringe assembly in accordance with embodiment 3 of the invention

### DESCRIPTION OF PREFERRED EMBODIMENTS

The present invention provides a device and the method of use for the delivery of fat graft material to treat delicate anatomical areas. By use of the device and method very low amounts of fat can be administered and easily be controlled.

This can be achieved by connecting the fat graft syringe assembly to a conventional delivery tubing system normally used for e.g. the controlled, low-volume application of local anesthetics. The driving force to move the fat particles through the syringe and the attached cannula is generated by a flow of normal saline solution. Normal saline flow can be controlled with a foot switch attached to an electronic infusion pump. This offers the ability to the surgeon to use his hand solely to control the position of the cannula-syringe system during the application procedure of the fat.

Thereby, this hand can achieve a stable position of the cannula that is not interfered by movement of a piston into the attached syringe—as in conventional injection syringes—by the same hand simultaneously.

As used herein, “fat graft material” refers to autogenous fat harvested from donor sites of the same human body by use of systems different to the syringe assembly embodiments described herein. After a specific treatment of the harvested fat graft material it is transferred into the syringe described below.

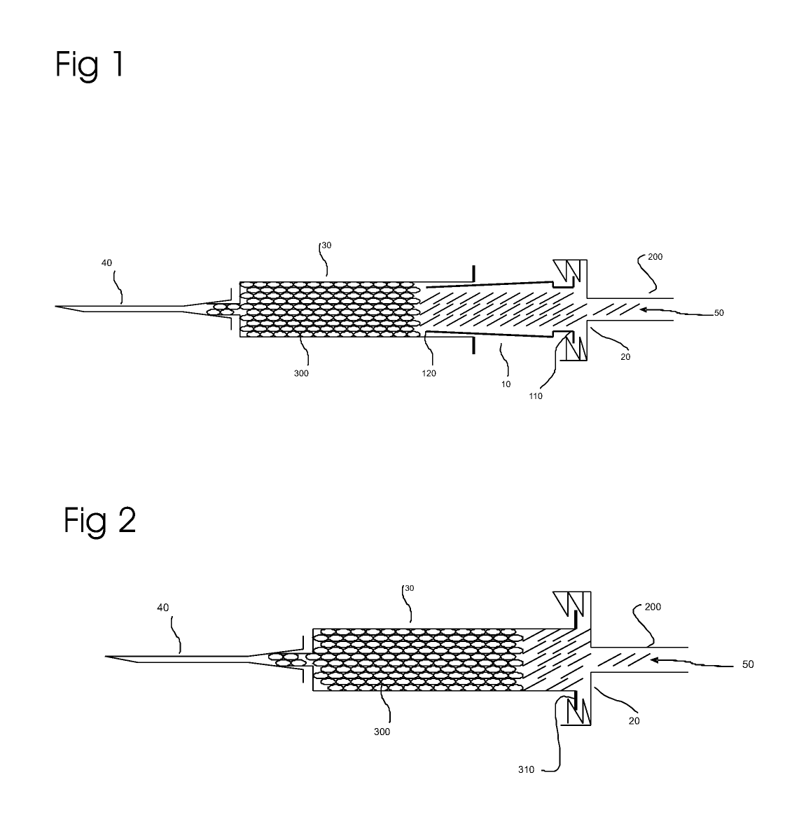

FIG. 1 is a side sectional view of the cylindrical fat graft syringe assembly in accordance with embodiment 1 of the invention.

10 includes the conical adapter part 120 and the external luer-lock part 110. 30 includes the syringe containing the graft material 300.

The attached internal luer-lock connector 20 enables the surgeon to take advantage of the driving force of normal saline 50 by compression through an electronic infusion pump (not shown). 40 is the injection cannula which is placed in the tissue.

Various connection subassemblies (including varying adapters and connectors) may be employed with the graft syringe assembly of the invention.

FIG. 2 is a side sectional view of the fat graft syringe assembly in accordance with embodiment 3 of the invention.

30 includes the syringe containing the graft material 300. The internal luer-lock thread 20 which connects directly to the syringe by an external luer-lock 310 enables the surgeon to take advantage of the driving force of normal saline 50 by compression through an electronic infusion pump (not shown).

Graft material is placed in the tissue by the injection cannula 40.
10 subassembly
110 external luer-lock
120 conical connection part
20 internal luer-lock connector of
200 injection line
30 syringe body
300 graft material
310 external luer-lock included in syringe body
40 injection needle
50 normal saline

In operation, when the surgeon activates the normal saline flow 50—by using a foot switch attached to an electronic injection pump—force is applied to the graft material 300 in the syringe 30. The normal saline entering the syringe forces the graft material from the inner chamber of the syringe 30 to leave the distal opening of the cannula 40 to the surgical site.

By use of the invention the surgeon's hand can guide the cannula through the tissue. Interference by a simultaneous activation of a piston being moved into the syringe using the same hand is avoided. The more delicate procedure—directing the needle—is made independent from the less delicate procedure—pushing the piston into the syringe. By use of an electronic injection device the less delicate procedure, however, can even be achieved slower and more accurately.

As described above, the graft material syringe assembly of the present invention offers the advantage of compatibility with a number of connection subassemblies.

It should be appreciated that the various embodiments of the invention described above may alternately use components of varying shape, size, diameter, cross-sectional configuration, as well as other components or fixtures known in the art.

As well graft material other than fat may also be used.

While the description above includes details and specificities, it is to be understood that these have been included to facilitate comprehension of the invention only, but it should be stressed that the invention is not limited to these.

Modifications to the embodiments described above can be made without changing the spirit and scope of the invention, which should be determined by the appended claims and their legal equivalents.

What is claimed is:

1. A graft syringe assembly for delivering fat graft material, comprising: a syringe subassembly including: a syringe adapter coupled to a syringe. The adapter consists of a proximal portion and a distal portion; the distal portion can be coupled to a 1 ml syringe by a conical fitting, the proximal portion of the adapter has an external luer-lock thread that can be affixed to an internal luer-lock thread of a conventional infusion, injection or local anesthetic delivery tubing system

2. The graft syringe assembly adapter of claim 1, wherein the proximal portion of the adapter has an internal luer-lock thread that can be affixed to an external luer-lock thread of a conventional infusion, injection or local anesthetic delivery tubing system

3. The graft syringe assembly of claim 1, wherein the syringe adapter is included in the 1 ml syringe body serving as its proximal connecting portion which has a size equal to an external luer-lock thread. Thereby, a conventional infusion or local anesthetic delivery tubing system with its distal internal luer-lock thread can be connected directly to the proximal portion of the syringe.

4. A method of combining the assemblies of claims 1-3 with a conventional electronic injection or infusion delivery system that contains normal saline and is activated electronically by a foot switch
